(19) United States
(12) Patent Application Publication
(10) Pub. No.: US 2025/0155419 A1
(43) Pub. Date: May 15, 2025

(54) FOOD PRODUCT SPOILAGE DETERMINATION METHOD, AND FOOD PRODUCT SPOILAGE DETERMINATION SYSTEM

(71) Applicant: Panasonic Intellectual Property Management Co., Ltd., Osaka (JP)

(72) Inventors: Yosuke HANAI, Osaka (JP); Atsuo NAKAO, Nara (JP); Masaya NAKATANI, Hyogo (JP)

(21) Appl. No.: 18/834,408

(22) PCT Filed: Feb. 3, 2023
(86) PCT No.: PCT/JP2023/003519

§ 371 Date: Jul. 30, 2024

(30) Foreign Application Priority Data
Feb. 3, 2022 (JP) 2022-015895

(51) Int. Cl. G01N 33/12; G01N 30/02; G01N 30/86

(52) U.S. Cl. CPC G01N 33/12; G01N 30/8693; G01N 2030/025

(57) ABSTRACT

A volatile component released from a food product is trapped. Detection data on the volatile component is acquired by using a detector. When the food product is spoiled, at least one of a type of the food product or a degree of spoilage of the food product is determined based on the detection data.

### TECHNICAL FIELD

The present disclosure relates to food product spoilage determination methods and food product spoilage determination systems and specifically relates to a food product spoilage determination method configured to determine, based on a gas released from a food product, spoilage of the food product and a food product spoilage determination system configured to implement the food product spoilage determination method.

### BACKGROUND ART

Patent Literature 1 discloses a sensor including a pH sensitive solution, the pH sensitive solution including methyl red and bromthymol blue mixed with an alkaline to have a pH value, the pH sensitive solution having a generally green color changing to a generally orange color responsive to exposure to a concentration of carbon dioxide, the sensor being configured to detect the presence of bacteria from a perishable food product. The solution is packaged in a gas permeable container using a TPX (PMP) thin film that allows an effective diffusion of carbon dioxide through the container. In this sensor, the pH level drops when acidic carbon dioxide comes into contact with the solution resulting from a formation of carbonic acid making the solution an indicator of carbon dioxide concentration and thus bacterial growth.

### CITATION LIST

### Patent Literature

Patent Literature 1: JP 2008-523391 A

### SUMMARY OF INVENTION

It is an object of the present disclosure to provide a food product spoilage determination method and a food product spoilage determination system which are configured to accurately determine spoilage of a food product.

A food product spoilage determination method according to an aspect of the present disclosure includes trapping a volatile component released from a food product, acquiring, by using a detector, detection data on a plurality of components included in the volatile component, and when the food product is spoiled, determining, based on the detection data, at least one of a type of the food product or a degree of spoilage of the food product.

A food product spoilage determination system according to an aspect of the present disclosure includes: a detector configured to detect a volatile component released from a food product to output an output signal; and a processor. The processor includes: an acquirer configured to acquire the output signal and generate, from the output signal, detection data on a plurality of components included in the volatile component; and a determining unit configured to, when the food product is spoiled, make, based on the detection data, a determination as to at least one of a type of the food product or a degree of spoilage of the food product.

### BRIEF DESCRIPTION OF DRAWINGS

FIG. 1 is a schematic system configuration diagram of a sensor device and a food product spoilage determination system according to an embodiment of the present disclosure;

FIG. 2 is a schematic illustrative view of a gas sensor included in the sensor device;

FIG. 3 is a chromatogram of a volatile component of ground pork, the chromatogram being obtained by using a gas chromatograph;

FIG. 4 is a chromatogram of a volatile component of ground chicken, the chromatogram being obtained by using a gas chromatograph;

FIG. 5 is a graph of a relationship between the number of days elapsed and a result of determining, based on detection data on a volatile component of ground pork, the degree of spoilage, the detection data being obtained by using the sensor device;

FIG. 6 is a graph of a relationship between the number of days elapsed and a result of determining, based on detection data on a volatile component of ground chicken, the degree of spoilage, the detection data being obtained by using the sensor device; and

FIG. 7 is a scatter diagram of pieces of detection data on volatile components of a plurality of samples including at least one of ground chicken or ground pork, the detection data being obtained by using the sensor device, where the abscissa (LD1) represents a first discriminant coefficient, and the ordinate (LD2) represents a second discriminant coefficient.

### DESCRIPTION OF EMBODIMENTS

First of all, circumstances under which the inventors developed the present disclosure will be briefly described.

For distribution and storage of food products, determining spoilage of the food products is very important from the viewpoint of safety management of the food products. For example, when a food product is spoiled due to an unexpected failure at the time of distribution and management of the food product and the presence of the failure is not realized, a spoiled food product may be provided to a demander. In such a case, if spoilage of the food product can be determined, the risk of the spoiled food product being provided to the demander can be reduced.

The technique described in Patent Literature 1 uses the sensor to indicate, based on carbon dioxide output from a food product, bacterial growth in the food product.

However, according to research conducted by the inventors, the amount of gas produced by metabolism of bacteria in a spoiled food product depends on the type of the food product. Moreover, when the amount of a food product is large, a large amount of gas is produced even with a low degree of spoilage, whereas when the amount of the food product is small, a small amount of gas is produced even with a high degree of spoilage. Therefore, simply detecting the carbon dioxide gas output from the food product as described in Patent Literature 1 cannot accurately determine the spoilage of the food product.

Thus, the inventors proceeded with the research and development to accurately determine the spoilage of the food product and have accomplished the present disclosure.

Note that the present disclosure has been accomplished under the circumstances described above, but the circumstances described above are not intended to limit the scope of the present disclosure. The scope of the present disclosure is set forth based on the configurations of the present disclosure.

Next, an embodiment of the present disclosure will be described.

A food product spoilage determination method according to the present embodiment includes trapping a volatile component released from a food product, acquiring, by using a detector, detection data on a plurality of components included in the volatile component, and when the food product is spoiled, determining, based on the detection data, at least one of a type of the food product or a degree of spoilage of the food product.

According to the research conducted by the inventors, the type and the amount of a gas produced by, for example, metabolism of bacteria when a food product is spoiled due to bacterial action depend on the type of the food product and depend also on the degree of spoilage of the food product. Thus, this is used to determine, based on the detection data obtained by detecting the plurality of components released from the food product, the spoilage of the food product, thereby accurately determining, when the food product is spoiled, the type of the food product and accurately determining the degree of spoilage of the food product.

Therefore, according to the present embodiment, determining the spoilage of the food product on the basis of the plurality of components enables the accuracy of determining the spoilage of the food product to be improved, and in particular, not only individually using amounts of the plurality of components but also using a mutual relationship between the amounts of the plurality of components to determine the spoilage of the food product enables the spoilage of the food product to be more accurately determined.

As the plurality of components (hereinafter referred to as marker components) which are detection targets in the volatile component, components whose released amount from the food product vary depending on the degree of spoilage of the food product are selected from components released from the food product. For example, a volatile component released from a food product is analyzed, and based on a result of the analysis, marker components can be specified.

Detection data obtained by using a detector 1 preferably includes information according to the amount of the marker components in the volatile component. In this case, data may directly represent the amount of the marker components or data does not have to directly represent the amount of the marker components. The detection data may be an output signal itself from the detector 1 or may be data generated from the output signal by A/D conversion or the like of the output signal.

The detector 1 preferably outputs an output signal according to the amount of the plurality of marker components. That is, the output signal from the detector 1 is preferably a signal according to the amount of the plurality of components included in the volatile component. The output signal may be a collection of a plurality of signals corresponding to respective amounts of the plurality of marker components or may be a signal which depends on the amounts of the plurality of marker components but which is not separated into signals corresponding to the respective amounts of the plurality of marker components. The detection data may be a collection of a plurality of pieces of information corresponding to respective amounts of the plurality of marker components or may be information which depends on the amounts of the plurality of marker components but which is not separated into pieces of information corresponding to the respective amounts of the plurality of marker components.

In this case, using the plurality of marker components enables the spoilage of the food product to be more accurately determined. In particular, not only individually using the amounts of the plurality of marker components but also using the mutual relationship between the amounts of the plurality of marker components to determine the spoilage of the food product enables the spoilage of the food product to be more accurately determined.

The detector 1 is not particularly limited as long as it outputs the output signal according to the amounts of the plurality of marker components. The aspect of the output signal is not limited as long as the output signal is a signal depending on the amount of the marker components. For example, the output signal may be a numerical value or may be a pattern such as a waveform.

The detector 1 includes, for example, a gas sensor 2. In this case, for example, a signal output from the gas sensor 2 when the gas sensor 2 is supplied with the volatile component is the output signal.

When the detector 1 includes the gas sensor 2, the gas sensor 2 may be a sensor array including a plurality of sensor elements Ax having sensitivity characteristics different from each other. In this case, the output signal is a collection of, for example, signals output from the plurality of sensor elements Ax. As in this case, when the gas sensor 2 is a sensor array, the spoilage of the food product can be determined based on a combination of a plurality of pieces of information, thereby increasing determination accuracy. Note that saying that the plurality of sensor elements Ax have sensitivity characteristics different from each other means that the plurality of sensor elements Ax are different from each other in terms of at least one of a detectable substance and a detection sensitivity to the substance. Moreover, each of the plurality of sensor elements Ax may have sensitivity to only one type of substance or may have sensitivity to two or more substances.

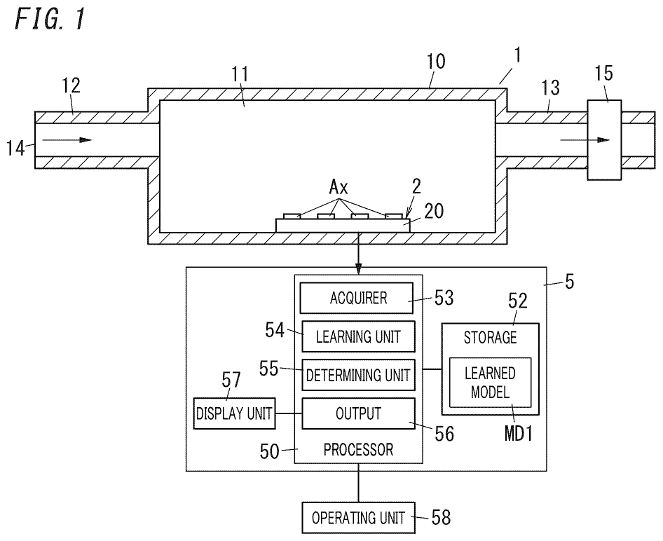

FIG. 1 shows an example of a sensor device which is the detector 1 including the gas sensor 2. FIG. 1 also shows a food product spoilage determination system 5 including the detector 1, but the food product spoilage determination system 5 will separately be described later, the sensor device will be described first.

The sensor device includes a sensor chamber 10, the gas sensor 2, a substrate 20, a suction port 14, an introduction path 12, an exhaust path 13, and a blower 15.

The sensor chamber 10 has an accommodation space 11 therein. To the sensor chamber 10, the introduction path 12 and the exhaust path 13 each of which is in communicative connection with the accommodation space 11 are connected. The introduction path 12 has a starting end having an opening as the suction port 14. The volatile component is supplied through the suction port 14 to the sensor device, which is the detector 1. The blower 15 produces a current of air which transports the volatile component to the sensor device, which is the detector 1. The blower 15 is, for example, an air pump or a fan. Operation of the blower 15 produces a current of air from the suction port 14 through the introduction path 12 to the accommodation space 11 and further toward the exhaust path 13. In FIG. 1, the blower 15 is disposed in the exhaust path 13 but may be disposed in, for example, the introduction path 12. When the blower 15 operates, the volatile component flows from the suction port 14 into the introduction path 12 and is introduced from the introduction path 12 into the accommodation space 11 in the sensor chamber 10, and the volatile component in the accommodation space 11 is further discharged outside from the accommodation space 11 through the exhaust path 13. The gas sensor 2 and the substrate 20 are housed in the accommodation space 11. In the accommodation space 11, the substrate 20 is disposed, and on the substrate 20, the gas sensor 2 is disposed. The rate of the current of air produced by the blower 15 is preferably, but not limited to being, greater than or equal to 10 mL/min and less than or equal to 3000 mL/min.

The gas sensor 2 outputs a signal according to the amount of marker components as described above. For example, the gas sensor 2 varies its electrical characteristic value in response to the marker components, and the change amount of the electrical characteristic value depends on the amount of the marker components.

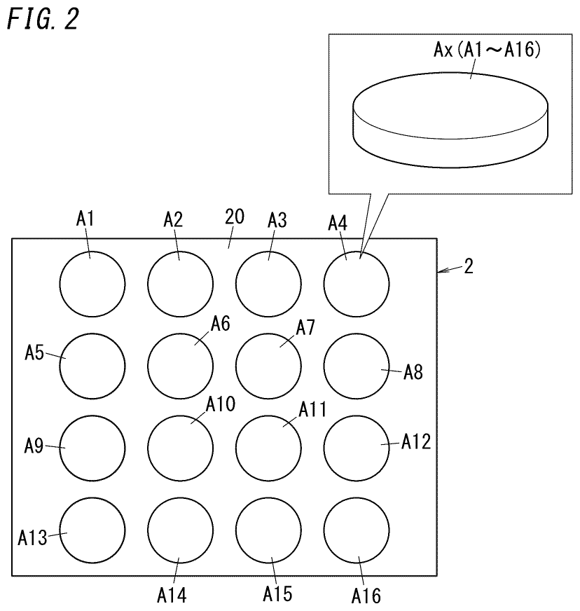

In the present embodiment, the gas sensor 2 is a sensor array including the plurality of sensor elements Ax having different sensitivity characteristics. In the present embodiment, the gas sensor 2 includes sixteen sensor elements Ax. The sixteen sensor elements Ax may be referred to as sensor elements A1 to A16 (see FIG. 2). The sixteen sensor elements A1 to A16 are disposed on the substrate 20 in four rows and four columns.

Each of the plurality of sensor elements Ax includes: a matrix including, for example, an organic material; and electrically conductive particles dispersed in the matrix. Each sensor element Ax shown in FIG. 2 is a film having a circular shape in plan view, but the shape of each sensor element Ax is not limited to this example.

As the organic material, a material having the property of adsorbing the marker components is selected. The organic material contains at least one selected from the group consisting of, for example, polydiethylene glycol adipate, diethylene glycol succinate, diglycerol, tetrahydroxy ethylene diamine, poly (ethylene glycol succinate), polyethylene glycol 4000 (manufactured by Sigma-Aldrich Co. LLC), polyethylene glycol 20000 (manufactured by Sigma-Aldrich Co. LLC), polyethylene glycol 20M (manufactured by Shinwa Chemical Industries Ltd.), free fatty acid polymer (manufactured by Shinwa Chemical Industries Ltd.), 1,2,3-tris (2-cyanoethoxy) propane, N,N-bis(2-cyanoethyl) formamide, Lac-3R-728 (manufactured by GL Sciences Inc.), Reoplex 400 (manufactured by Shinwa Chemical Industries Ltd.), SP-2330 (manufactured by Sigma-Aldrich Co. LLC), SP-2340 (manufactured by Sigma-Aldrich Co. LLC), and UCON 75-HB-90000 (manufactured by Shinwa Chemical Industries Ltd.). These materials have the characteristic of adsorbing various components and have adsorption performances different from each other and can thus be used to detect the plurality of marker components.

When the gas sensor 2 includes the plurality of sensor elements Ax, if the plurality of sensor elements Ax include organic materials different from each other, the plurality of sensor elements Ax can have sensitivity characteristics different from each other. As long as the organic material has the property of adsorbing the marker components, the organic material is not limited to the examples described above.

The conductive particles include at least one type of material selected from the group consisting of, for example, a carbon material, a conductive polymer, metal, a metal oxide, a semiconductor, a superconductor, and a complex compound. The carbon material includes at least one type of material selected from the group consisting of, for example, carbon black, graphite, coke, a carbon nanotube, graphene, and fullerene. The conductive polymer includes at least one type of material selected from the group consisting of, for example, polyaniline, polythiophene, polypyrrole, and polyacetylene. The metal includes at least one type of material selected from the group consisting of, for example, silver, gold, copper, platinum, and aluminum. The metal oxide includes at least one type of material selected from the group consisting of, for example, indium oxide, tin oxide, tungsten oxide, zinc oxide, and titanium oxide. The semiconductor includes at least one type of material selected from the group consisting of, for example, silicon, gallium arsenide, indium phosphide, and molybdenum sulfide. The superconductor includes at least one type of material selected from the group consisting of, for example, YBa₂Cu₃O₇ and Tl₂Ba₂Ca₂Cu₃O₁₀. The complex compound includes at least one type of material selected from the group consisting of, for example, a complex compound of tetramethyl-para-phenylenediamine and chloranil, a complex compound of tetracyanoquinodimethan and alkali metal, a complex compound of tetrathiafulvalene and halogen, a complex compound of iridium and a halocarbonyl compound, and tetracyano platinum.

When the organic material in each sensor element Ax adsorbs the marker components, the volume of the matrix increases, thereby increasing the distance between the conductive particles in each sensor element Ax. Accordingly, the electrical resistance value of each sensor element Ax increases. As the amount of the marker components adsorbed on the organic material increases, the electrical resistance value of each sensor element Ax increases. Therefore, a change in the electrical resistance value of each sensor element Ax depends on the amount of the marker components.

The substrate 20 includes an electrode connected to each sensor element Ax. When a voltage is applied from the electrode to the sensor elements Ax, currents according to respective electrical resistance values of the sensor elements Ax flow to the sensor elements Ax. For example, the currents according to the respective electrical resistance values are signals output from the sensor elements Ax. A collection of the currents output from the sensor elements Ax is the output signal from the sensor device.

The detector 1 may be a gas chromatograph. In this case, for example, a chromatogram output from the gas chromatograph when the volatile component is supplied to the gas chromatograph is the output signal.

The detector 1 may be an appropriate means other than the examples above.

The aspect of the detector 1 when the detector 1 includes the gas sensor is not limited to the example above. For example, the aspect of the gas sensor is not limited to the example above. For example, the intensity of, or the amount of change in: the weight of, the electrical characteristic (e.g., the electrical resistance value or the permittivity) of, the resonance frequency of, the quantity of light output from, or the quantity of radiation from, the gas sensor, for example, when marker components are adsorbed on, are bound to, are trapped in, or interact with an appropriate gas sensor may be output as the output signal.

The detector 1 may be a device which measures the weight of the marker components after the marker components in the volatile component are liquefied or solidified by, for example, condensation.

The detector 1 may be a device which quantitates the marker components by measuring the absorbance of the marker components in the volatile component.

The detector 1 may be a device which outputs, as the output signal, a signal obtainable from the gas detector when the volatile component is directly, or the volatile component held in an adsorption tube is, introduced into a measuring instrument equipped with the gas detector. In the measuring instrument, a separation device may be disposed upstream of the detector. The separating device is, for example, a capillary column for separating the marker components from the volatile component. An example of the detector 1 in this case is the gas chromatograph described above. The detector is, for example, a detector based on catalytic oxidation non-dispersive infrared absorption (NDIR), a hydrogen flame ionization detector (FID), a photoionization detector (PID), a mass spectrograph (MS), or a semiconductor gas sensor.

The detector 1 may include a detector tube. The detector tube is, for example, a glass tube which is densely filled with a detection agent reactive to the marker components and which has a surface provided with a scale. When the volatile component is introduced into the detector tube, part of the detection agent reacts with the marker components and discolors. The degree of discoloring of the detection agent is the detection data. For example, the length of a discolored part of the detection agent is determined by reading the scale, and based on this length, the marker components introduced into the detector tube can be quantitated.

Based on the detection data on the plurality of components (marker components) included in the volatile component, the spoilage of the food product is determined. To determine the spoilage of the food product on the basis of the detection data, for example, a combination of the detection data on, and the degree of spoilage of, a food product whose degree of spoilage is known is accumulated as learning data. The degree of spoilage can be defined by an arbitrary method. For example, the degree of spoilage may be defined in association with an elapsed time when a specified type of food product is disposed in a certain environment in which the spoilage of the food product can progress. Specifically, for example, the degree of spoilage at a time point at which one day has elapsed since a food product was disposed in a certain environment may be defined as “1”, the degree of spoilage at a time point at which two days have elapsed since the food product was disposed in the certain environment may be defied as “2”, and the like.

This learning data is used to create a learned model for determining the spoilage on the basis of the detection data. Thus, using this learned model enables the type and the degree of spoilage of the food product to be determined, for example, on the basis of the detection data on a food product whose type and degree of spoilage are unknown. To create the learned model, for example, a program (algorithm) of artificial intelligence is caused to perform machine learning using the learning data, thereby generating the learned model. The program of the artificial intelligence is a model of the machine learning, and is, for example, a random forest or a neural network.

To make a determination by using the learned model, the determination preferably includes a discriminant analysis based on training data (teaching data) and the detection data. In this case, for example, the determination preferably includes a discriminant analysis based on the distance or similarly between the teaching data and the detection data. Moreover, the determination preferably includes a linear discriminant analysis based on the teaching data and the detection data.

The food product spoilage determination system 5 (hereinafter referred to also as a determination system 5) will be described. The determination system 5 implements the food product spoilage determination method. The determination system 5 includes: the detector 1; and a determining unit 55 configured to determine, based on the detection data output from the detector 1, the spoilage of the food product.

FIG. 1 shows an overview of a configuration example of the determination system 5 including a sensor device as the detector 1. Note that as already described, the detector 1 is not limited to the sensor device.

In the example shown in FIG. 1, the determination system 5 includes: a processor 50 including the determining unit 55; a storage 52; a display unit 57; and an operating unit 58.

The processor 50 is a control circuit configured to control the operation of the determination system 5. The processor 50 may be implemented by, for example, a computer system including one or more processors (microprocessors) and one or more memory elements. That is, the one or more processors execute one or more programs (applications) stored in the one or more memory elements, thereby functioning as the processor 50. The program(s) is stored in the memory element(s) of the processor 50 or the storage 52 in advance in this embodiment but may be provided over a telecommunications network such as the Internet, or may be provided as a non-transitory recording medium such as a memory card in which the program(s) has been stored.

As shown in FIG. 1, the processor 50 includes an acquirer 53, a learning unit 54, and an output 56 in addition to the determining unit 55. The acquirer 53, the learning unit 54, the determining unit 55, and the output 56 do not represent tangible components but show functions implemented by the processor 50.

The acquirer 53 acquires the output signal output from the sensor device, which is the detector 1, and the acquirer 53 performs, for example, conversion of the output signal into digital data, thereby generating the detection data from the output signal.

The learning unit 54 causes a program (algorithm) of artificial intelligence to perform machine learning using learning data to generate a learned model, and the learning unit 54 causes the storage 52 to store the learned model. That is, the learning unit 54 is responsible for a learning phase in a preparation stage before determining the spoilage by using the determination system 5. In the learning phase, a combination of detection data on, and the type and the degree of spoilage of, a food product whose type and degree of spoilage are unknown is accumulated as the learning data in the storage 52, and a learned model MD1 is created from the learning data. Note that the learning unit 54 may perform re-learning using learning data newly collected by the acquirer 53 after generation of the learned model MD1 to improve the performance of the learned model MD1.

When the food product is spoiled, the determining unit 55 uses the learned model MD1 stored in the storage 52 to determine, based on the detection data, at least one of the type of the food product or the degree of spoilage of the food product. Determining the type of the food product when the food product is spoiled means, for example, determining the type of the food product and determining whether or not the food product is spoiled. Determining the degree of spoilage of the food product means determining the degree of the degree of spoilage of the food product in a manner recognizable to humans. For example, the determining unit 55 may select a numerical value corresponding to the degree of spoilage of the food product to determine the degree of spoilage of the food product, may select a color corresponding to the degree of spoilage of the food product to determine the degree of spoilage of the food product, or may select wording corresponding to the degree of spoilage of the food product to determine the degree of spoilage of the food product.

The output 56 outputs a determination result of spoilage determination by the determining unit 55 to the display unit 57.

The storage 52 includes one or more storage devices. The storage device(s) is, for example, RAM, ROM, or EEPROM. The storage 52 stores the learned model MD1 and the like. The learned model MD1 may be generated in the learning phase in which the determination system 5 is used as described above, but the learned model MD1 may be generated by a learning system other than the determination system 5. When the learned model MD1 is generated by a learning system other than the determination system 5, the determination system 5 does not have to include the learning unit 54.

The display unit 57 displays the determination result output from the output 56 externally in a manner recognizable to humans. The display unit 57 is, for example, a device which visibly displays a determination result of spoilage, and in this case, the display unit 57 includes, for example, a display device such as a liquid crystal display. The display unit 57 may be a device which expresses a determination result of spoilage determination by using audio, and in this case, the display unit 57 includes, for example, a buzzer or a loudspeaker. The operating unit 58 receives an operation given by a user and causes the processor 50 to operation in accordance with the operation given by the user. The operating unit 58 includes, for example, a switch, keyboard, touch panel, or speech recognition device for receiving the operation given by the user.

An example of operation of the determination system 5 in an inference phase will be described. Note that the operation in the inference phase is a mere example as described above, and the order of processes may be changed, or a process(es) may accordingly be added or omitted.

A user, for example, gives an operation to the operating unit 58 to activate the processor 50, thereby causing the determination system 5 to start the operation of determining the spoilage. The user introduces a volatile component released from a food product through the introduction path 12 into the accommodation space 11 to expose the gas sensor 2 to the volatile component (exposition step).

The acquirer 53 acquires an output signal from the detector 1 to generate detection data from the output signal (acquisition step). The determining unit 55 inputs the detection data generated by the acquirer 53 to the learned model MD1, thereby determining the spoilage of the food product (determination step).

When the spoilage of the food product is determined by the determining unit 55, the output 56 outputs the determination result by the determining unit 55 to the display unit 57 (output step). Thus, the user can confirm the determination result by reviewing the displayed content of the display unit 57.

In the present embodiment, to determine the spoilage of a food product, for example, a volatile component released from a specified type of food product is trapped, the volatile component is detected by using the detector 1, detection data on the plurality of components included in the volatile components is generated from an output signal of the detector 1, and the degree of spoilage of the specified type of food product is determined based on the detection data. In this case, the type of the food product is specified in advance, and therefore, a determination as to the type of a spoiled food product does not have to be made. Of course, the determination as to the type of the spoiled food product may be made. In this case, the determination can be made, for example, by using a learned model created by using learning data on the specified type of food product.

In the present embodiment, to determine the spoilage of a food product, for example, a volatile component released from an unknown type of food product may be trapped, the volatile component may be detected by using the detector 1, detection data on a plurality of components included in the volatile component may be acquired from an output signal of the detector 1, and the type of the food product which is spoiled and the degree of spoilage of the food product may be determined based on the detection data. In this case, the determination may be made, for example, by using a learned model created by using learning data on a plurality of food products assumed to include the unknown type of food product.

Moreover, in the present embodiment, in a situation where a plurality of food products are present, a gas in an atmosphere surrounding the plurality of food products may be trapped, the volatile component may be detected by using the detector 1, detection data on a plurality of components included in the gas may be generated from an output signal from the detector 1, and when a food product of the plurality of food products is spoiled, the type of the spoiled food product may be determined based on the detection data. The degree of spoilage of the spoiled food product may be further determined. If two or more food products of the plurality of food products are spoiled, the type of each of the two or more spoiled food products may be determined, and additionally, the degree of spoilage of each of the two or more spoiled food products may be determined. In this case, the determination may be made, for example, by using the learned model created by using the learning data on each of the plurality of food products.

The food product spoilage determination method and the food product spoilage determination system according to the present embodiment are applicable to various situations in which food products are handled. For example, they are applicable to an inspection of food products before shipment to food product factories and the like, a freshness inspection of the food products received in the food product factories and the like, and home-use refrigerators.

The embodiment described above is a mere example of various embodiments of the present disclosure. The embodiment described above may be modified in various manners depending on the design or the like as long as the object of the present disclosure is achieved. Variations of the embodiment will be described below. Any of the variations to be described below may be combined as appropriate.

The determination system 5 of the present disclosure includes a computer system in the processor 50 or the like. The computer system may include a processor and a memory element as principal hardware components thereof. The processor executes a program stored in the memory element of the computer system, thereby implementing the function as the determination system 5 in the present disclosure. The program may be stored in advance in the memory element of the computer system. Alternatively, the program may also be downloaded over a telecommunications network or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits include a system LSI, a very-large-scale integrated circuit (VLSI), and an ultra-large-scale integrated circuit (ULSI). Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be integrated together in a single device or distributed in multiple devices without limitation. As used herein, the “computer system” includes a microcontroller including one or more processors and one or more memory elements. Thus, the microcontroller may also be implemented as a single or a plurality of electronic circuits including a semiconductor integrated circuit or a large-scale integrated circuit.

Moreover, collecting the plurality of functions in the determination system 5 in a single housing is not an essential configuration for the determination system 5. The components of the determination system 5 may be distributed in a plurality of housings. Further, at least some functions of the determination system 5 (e.g., some functions of the determination system 5) may be implemented as a cloud computing system as well.

The plurality of functions in the determination system 5 may be, for example, collected in a single housing so that the determination system 5 configures a single device. In this case, the determination system 5 can be brought to a site where a food product is stored, thereby facilitating an on-site determination as to the spoilage of the food product.

In the determination system 5 of the embodiment described above, the gas sensor 2 includes sixteen sensor elements Ax, but the number of sensor elements Ax is accordingly variable. Moreover, in the determination system 5 of the embodiment described above, the sixteen sensor elements Ax are arranged in four rows and four columns, but the arrangement of the plurality of sensor elements Ax is not limited to the embodiment described above. The plurality of sensor elements may be arranged to be aligned in a line or may be arranged in one circle or two or more concentric circles with intervals.

In the determination system 5 of the embodiment described above, the learned model MD1 is stored in the storage 52 of the determination system 5, but the determination system 5 may determine the spoilage of the food product by using the learned model MD1 disposed in a cloud system. That is, the determining unit 55 of the determination system 5 may input the detection data output from the detector 1 to a learned model in the cloud system and may acquire a determination result from the learned model in the cloud system to determine the spoilage of the food product.

In the present embodiment, the types of food products are not limited. In the present disclosure, appropriate marker components are selected by, for example, analyzing the volatile component in advance in accordance with the type of a food product, and then, the spoilage of the food product can be determined. Moreover, also when the marker components are not definitely specified, it is required only that the detection data output from the detector 1 is information depending on the amount of the plurality of components in the volatile component, the amount correlating with the degree of spoilage of the food product.

For example, when the food product includes pork, the marker components include preferably at least one, more preferably two or more, selected from the group consisting of 1-decanol (CAS number 112-30-1), 2-butanone (CAS number 78-93-3), ethyl 2-methylbutyrate (CAS number 7452-79-1), 2-pentanone (CAS number 107-87-9), dimethyl disulfide (CAS number 624-92-0), dimethyl trisulfide (CAS number 3658-80-8), ethyl tiglate (CAS number 5837-78-5), isobutyl alcohol (78-83-1), trimethylamine (CAS number 75-50-3), 2-heptanone (CAS number 110-43-0), and isobutyl isobutyrate (CAS number 97-85-8). When the volatile component is analyzed by gas chromatography-mass spectrometry, it can be confirmed that the proportion of these components in the volatile component varies in accordance with the degree of spoilage of the pork, and therefore, these components can be used to accurately determine the spoilage the pork. The marker components particularly preferably include at least one of the 2-heptanone or the isobutyl isobutyrate. In this case, the amount of each of the 2-heptanone and the isobutyl isobutyrate strongly correlates with, in particular, the degree of spoilage of the pork, and therefore, the spoilage of the pork can be more accurately determined. Note that a result of the analysis of the volatile component of the pork will be described later in Examples.

Moreover, for example, when the food product includes chicken, the marker components include at least one, more preferably two or more, selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, anisole (CAS number 100-66-3), and styrene (CAS number 100-42-5). When the volatile component is analyzed by gas chromatography-mass spectrometry, it can be confirmed that the proportion of these components in the volatile component varies in accordance with the degree of spoilage of the chicken, and therefore, these components can be used to accurately determine the spoilage of the chicken. The marker components particularly preferably include at least one of the anisole or the styrene. In this case, the amount of each of the anisole and the styrene strongly correlates with, in particular, the degree of spoilage of the chicken, and therefore, the spoilage of the chicken can be more accurately determined. Note that a result of the analysis of the volatile component of the chicken will be described later in Examples.

When the food product includes both pork and chicken, the marker components include preferably at least one, more preferably two or more, selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, 2-heptanone, isobutyl isobutyrate, anisole, and styrene. When the volatile component is analyzed by gas chromatography-mass spectrometry, it can be confirmed that the proportion of these components in the volatile component varies in accordance with one of, or both, the degree of spoilage of the pork and the degree of spoilage of the chicken, and therefore, these components can be used to accurately determine the spoilage the pork. The marker components particularly preferably include at least one of the 2-heptanone or the isobutyl isobutyrate and at least one of the anisole or the styrene. In this case, the amount of each of the 2-heptanone and the isobutyl isobutyrate strongly correlates with, in particular, the degree of spoilage of the pork but weakly correlates with the degree of spoilage of the chicken, and the amount of each of the anisole and the styrene strongly correlates with, in particular, the degree of spoilage of the chicken but weakly correlates with the degree of spoilage of the pork. Therefore, whether the spoiled food product is pork or chicken can be determined, and further, the degree of spoilage of each of the pork and the chicken can also be determined.

### Examples

As examples, a result of verifying that the food product spoilage determination method and the food product spoilage determination system according to the present embodiment enable the spoilage of a food product to be determined is shown below.

### 1. Verification of Use of Gas Chromatograph as Detector

Fresh ground pork and fresh ground chicken were prepared.

The ground pork was settled in a polypropylene container at a room temperature (about 25° C.). On each of day 0 (which is a time point at which the ground pork was put in the polypropylene container), and day 1 (a time point at which 24 hours had elapsed), day 2 (a time point at which 48 hours had elapsed), and day 3 (a time point at which 72 hours had elapsed) since the time point at which the ground pork was put in the polypropylene container, a gas (200 mL) including a volatile component released from the ground pork was introduced into an adsorption tube in which an adsorption agent (tenax GR) had been put and was trapped by being adsorbed onto the adsorption agent. The adsorption tube was heated to desorb the gas from the adsorption agent, and the gas was analyzed by using a gas chromatograph mass spectrometer (manufactured by Shimadzu Corporation, GCMS-QP2010 Ultra) under the following conditions.

The ground chicken was subjected to the same test.

a. Configuration of Gas Chromatograph
Column: InertCap 5 MS/Sil (0.25 mm inner diameter, 30 m length, 1 μm thickness)
Inlet: OPTIC4
Autosampler: AOC-5000
   b. Supply Condition of Gas to Column
Heating temperature of adsorption tube: The temperature of the adsorption tube was increased from 35° C. to 250° C. at a rate of temperature rise of 20° C./sec, and was kept at 250° C. for 3 minutes.
Injection mode: Splitless
Carrier gas: Helium
Flow rate: 1 ml/min
Column heating condition: The temperature of the column was kept at 30° C. for 10 minutes, was subsequently increased to 100° C. at a rate of temperature rise of 2° C./min, was subsequently increased to 200° C. at a rate of temperature rise of 4° C./min, was subsequently increased to 250° C. at a rate of temperature rise of 10° C./min, and was subsequently kept at 250° C. for 5 minutes.
   c. Mass Spectroscopy Condition
Ionization: EI
Ion source temperature: 200° C.
m/z range: m/z35-300 (Scan mode)
Interface temperature: 200° C.

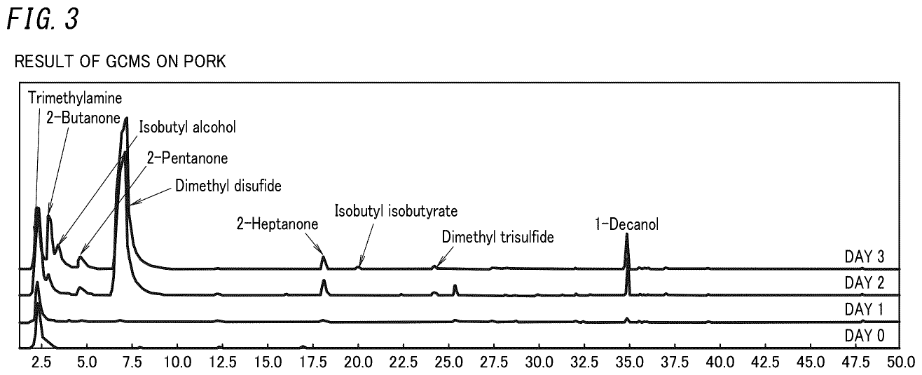

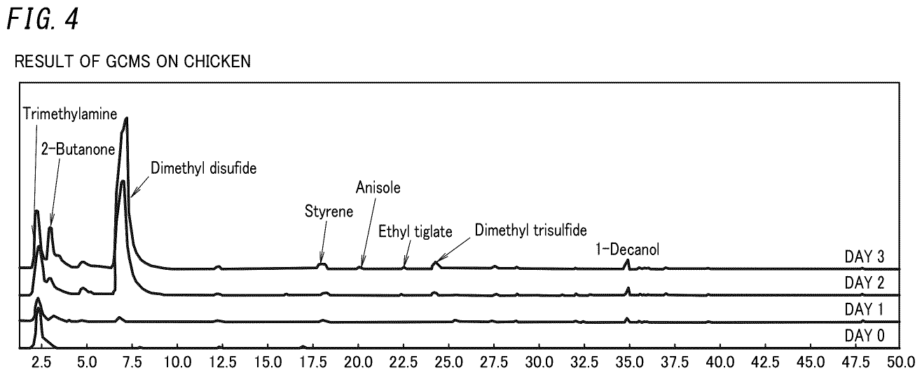

FIG. 3 shows a chromatogram obtained for the ground pork. FIG. 4 shows a chromatogram obtained for the ground chicken.

According to FIG. 3, peaks are observed in the chromatogram obtained for the ground pork, and the peaks correspond to, for example, 1-decanol (holding time 35.06 minutes), 2-butanone (holding time 3.20 minutes), ethyl 2-methylbutyrate (holding time 15.00 minutes), 2-pentanone (holding time 4.93 minutes), dimethyl disulfide (holding time 7.09 minutes), dimethyl trisulfide (holding time 24.45 minutes), ethyl tiglate (holding time 22.65 minutes), isobutyl alcohol (holding time 3.73 minutes), trimethylamine (holding time 2.44 minutes), 2-heptanone (holding time 18.34 minutes), and isobutyl isobutyrate (holding time 20.65 minutes). Moreover, the intensity of each peak changed as days elapsed. Therefore, two or more components selected from these components can be used to determine the spoilage of the ground pork.

Moreover, according to FIG. 4, peaks are observed in the chromatogram obtained for the ground chicken, and the peaks correspond to, for example, 1-decanol (holding time 35.06 minutes), 2-butanone (holding time 3.20 minutes), ethyl 2-methylbutyrate (holding time 15.00 minutes), 2-pentanone (holding time 4.93 minutes), dimethyl disulfide (holding time 7.09 minutes), dimethyl trisulfide (holding time 24.45 minutes), ethyl tiglate (holding time 22.65 minutes), isobutyl alcohol (holding time 3.73 minutes), trimethylamine (holding time 2.44 minutes), anisole (holding time 20.34 minutes), and styrene (holding time 18.16 minutes). Moreover, the intensity of each peak changed as days elapsed. Therefore, two or more components selected from these components can be used to determine the spoilage of the ground chicken.

Moreover, according to the result above, it can be confirmed that in both the cases of the ground pork and the ground chicken, the amount of the plurality of components included in the volatile component changes as the spoilage progresses. Therefore, the degree of spoilage can be determined based on the waveform of the chromatogram.

Moreover, the waveform of the chromatogram of the ground pork and the waveform of the chromatogram of the ground chicken are different from each other. In particular, in the chromatogram of the ground pork, peaks of 2-heptanone and isobutyric acid isobutyl ether can be confirmed, whereas in the chromatogram of the ground chicken, none of these peaks can be confirmed. Moreover, in the chromatogram of the ground chicken, peaks of styrene and methoxy benzene can be confirmed, whereas none of these peaks is confirmed in the chromatogram of the ground pork. Thus, when whether the food product is ground pork or ground chicken is unknown, and the ground pork and the ground chicken are put in the same container, or the like, analyzing the volatile component by gas chromatograph enables whether the spoiled food product is the ground pork or the ground chicken to be determined based on the chromatogram thus obtained.

### 2. Verification of Use of Sensor Device as Detector

### (1) Verification of Individual Food Product

A sensor device was prepared which includes a gas sensor (sensor array) including sixteen sensor elements including, as their respective organic materials, polydiethylene glycol adipate, diethylene glycol succinate, diglycerol, tetrahydroxy ethylene diamine, poly (ethylene glycol succinate), polyethylene glycol 4000 (manufactured by Sigma-Aldrich Co. LLC), polyethylene glycol 20000 (manufactured by Sigma-Aldrich Co. LLC), polyethylene glycol 20M (manufactured by Shinwa Chemical Industries Ltd.), free fatty acid polymer (manufactured by Shinwa Chemical Industries Ltd.), 1,2,3-tris (2-cyanoethoxy) propane, N,N-bis(2-cyanoethyl) formamide, Lac-3R-728 (manufactured by GL Sciences Inc.), Reoplex 400 (manufactured by Shinwa Chemical Industries Ltd.), SP-2330 (manufactured by Sigma-Aldrich Co. LLC), SP-2340 (manufactured by Sigma-Aldrich Co. LLC), and UCON 75-HB-90000 (manufactured by Shinwa Chemical Industries Ltd.).

Ground pork the same as that used in “1. Verification of Use of Gas Chromatograph as Detector” described above was settled in a polypropylene container at a room temperature (about 25° C.). On each of day 0 (after 0 hours), day 1 (after 24 hours), day 2 (after 48 hours), day 3 (after 72 hours), and day 4 (after 96 hours) from a time point at which the ground pork was put in the polypropylene container, a volatile component was trapped. While a voltage was applied to each sensor element of the sensor device, each sensor element was exposed to the volatile component for 6 seconds, and the gas sensor was then exposed to clean air for 18 seconds. This process was repeated ten times. A collection of changes in a current flowing through each sensor element during this period was obtained as an output signal, and from the output signal, the detection data was generated.

The ground chicken was subjected to the same test.

The elapsed time since the time point at which the food product was put in the polypropylene container at a room temperature (about 25° C.) was defined as an index of the degree of spoilage, a combination of the detection data on, and the degree of spoilage of, the ground pork and a combination of the detection data on, and the degree of spoilage of, the ground chicken were accumulated as learning data, and from the learning data, a learned model was created by a random forest method. The learned model is an algorithm for determining, based on the detection data, the type of the food product and the degree of spoilage of the food product. Note that to create the learned model, a classifier was configured by using part of the learning data as teaching data, and the remaining learning data was used as test data.

From the detection data included in the test data, the type of the food product and whether or not the food product is spoiled were determined by using the learned model.

As a result, when an actual elapsed time for the detection data corresponds to one day (24 hours), based on four pieces of detection data on spoiled ground pork, the type of the food product was determined to be ground pork and the food product was determined to be spoiled. Moreover, based on eleven pieces of detection data on unspoiled ground pork, the type of the food product was determined to be ground pork, and the food product was determined to be unspoiled. Moreover, based on seven pieces of detection data on spoiled ground chicken, the type of the food product was determined to be ground chicken, and the food product was determined to be spoiled. Moreover, based on six pieces of detection data on unspoiled ground chicken, the type of the food product was determined to be ground chicken, and the food product was determined to be unspoiled. That is, the type of the food product and whether or not the food product is spoiled were determined with 100% accuracy.

Moreover, when the actual elapsed time for the detection data corresponds to two days (48 hours), based on nine pieces of detection data on spoiled ground pork, the type of the food product was determined to be ground pork and the food product was determined to be spoiled. Moreover, based on eight pieces of detection data on unspoiled ground pork, the type of the food product was determined to be ground pork and the food product was determined to be unspoiled. Moreover, based on eleven pieces of detection data on unspoiled ground chicken, the type of the food product was determined to be ground chicken and the food product was determined to be spoiled. Moreover, based on six pieces of detection data on unspoiled ground chicken, the type of the food product was determined to be ground chicken, and the food product was determined to be unspoiled. That is, the type of the food product and whether or not the food product is spoiled were determined with 100% accuracy.

Moreover, when the actual elapsed time for the detection data corresponds to three days (72 hours), based on six pieces of detection data on spoiled ground pork, the type of the food product was determined to be ground pork and the food product was determined to be spoiled. Moreover, based on six pieces of detection data on unspoiled ground pork, the type of the food product was determined to be ground pork and the food product was determined to be unspoiled. Moreover, based on nine pieces of detection data on unspoiled ground chicken, the type of the food product was determined to be ground chicken and the food product was determined to be spoiled. Moreover, based on eight pieces of detection data on unspoiled ground chicken, the type of the food product was determined to be ground chicken, and the food product was determined to be unspoiled. That is, the type of the food product and whether or not the food product is spoiled were determined with 100% accuracy.

Moreover, when the actual elapsed time for the detection data corresponds to four days (96 hours), based on six pieces of detection data on spoiled ground pork, the type of the food product was determined to be ground pork and the food product was determined to be spoiled. Moreover, based on ten pieces of detection data on unspoiled ground pork, the type of the food product was determined to be ground pork, and the food product was determined to be unspoiled. Moreover, based on six pieces of detection data on spoiled ground chicken, the type of the food product was determined to be ground chicken, and the food product was determined to be spoiled. Moreover, based on seven pieces of detection data on unspoiled ground chicken, the type of the food product was determined to be ground chicken, and the food product was determined to be unspoiled. That is, the type of the food product and whether or not the food product is spoiled were determined with 100% accuracy.

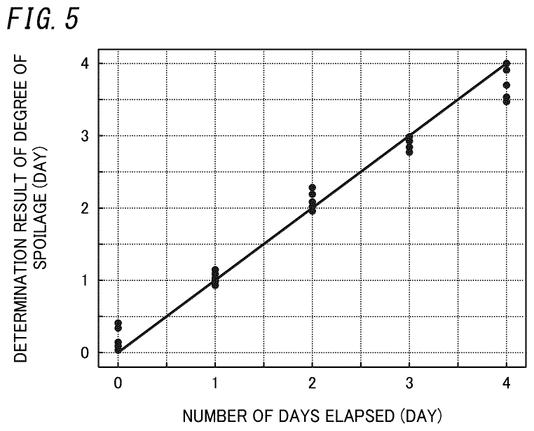

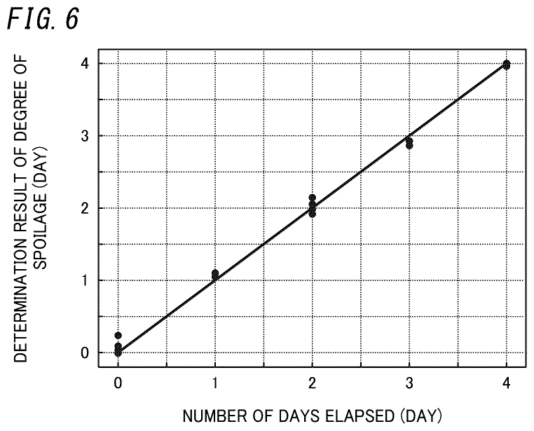

Moreover, based on each of the detection data on the ground pork and the detection data on the ground chicken, the degree of spoilage was determined by using the learned model. The determination result for the ground pork is shown in FIG. 5. The determination result for the ground chicken is shown in FIG. 6. In each of the figures, the ordinate shows the determination result by using the learned model with respect to the elapsed time, and the abscissa shows the actual elapsed time for the detection data.

As shown in FIGS. 5 and 6, the degree of spoilage determined based on the detection data substantially corresponds to the actual elapsed time.

The calculation of accuracy of the determination result for the ground pork resulted in a mean absolute error of 0.141, a root mean squared error (RMSE) of 0.193, and a coefficient of determination (R²) of 0.981. Moreover, the calculation of accuracy of the determination result for the ground chicken resulted in a mean absolute error of 0.049, a root mean squared error (RMSE) of 0.073, and a coefficient of determination (R²) of 0.997. Thus, it was confirmed that the determination was made with high accuracy in any case.

### (2) Verification in Case of Presence of Plurality of Food Products

A sensor device the same as that in “1. Verification of Individual Food Product” was prepared.

Moreover, the following samples were prepared.
Sample 1:20 g of fresh ground chicken
Sample 2:20 g of fresh ground pork
Sample 3:20 g of spoiled ground chicken
Sample 4:20 g of spoiled ground pork
Sample 5:10 g of spoiled ground chicken and 10 g of fresh ground pork
Sample 6:10 g of fresh ground chicken and 10 g of spoiled ground pork
Sample 7:10 g of spoiled ground chicken and 10 g of spoiled ground pork

Note that the spoiled ground chicken was obtained by putting fresh ground chicken in a polypropylene container at a room temperature (about 25° C.) and allowing to stand for two days to be spoiled. Moreover, the spoiled ground pork was obtained by putting fresh ground pork in a polypropylene container at a room temperature (about 25° C.) and allowing to stand for two days to be spoiled.

A learned model was created by the following method. A volatile component trapped from each of the seven samples was measured by using the sensor device. While a voltage was applied to each sensor element of the sensor device, each sensor element was exposed to the volatile component for 6 seconds, and the gas sensor was then exposed to clean air for 18 seconds. This process was repeated ten times. A collection of changes in a current flowing through each sensor element during this period was obtained as an output signal, and from the output signal, the detection data was generated. To create the learned model, the seven samples were classified into five groups shown in Table 1 below, and for each of the five groups, a combination of the detection data and the type and degree of spoilage of the food product were collected as learning data. From the learning data, a learned model which is an algorithm for determining, based on the detection data, the type of the food product and the degree of spoilage of the food product was created by linear discriminant analysis. Note that to create the learned model, part of the learning data was used as teaching data to configure the classifier, and the remaining learning data was used as test data.

TABLE 1

| | Group | |
|---|---|---|
| Symbol | Name | Description of Group |
| ⋄ | Group 1 | Including only fresh ground chicken (Sample 1) |
| □ | Group 2 | Including only fresh ground pork (Sample 2) |
| Δ | Group 3 | Including spoiled ground chicken but not including |
| | | spoiled ground pork (Sample 3 and Sample 5) |
| X | Group 4 | Including spoiled ground pork but not including |
| | | spoiled ground chicken (Sample 4 and Sample 6) |
| ◯ | Group 5 | Including spoiled ground pork and spoiled ground |
| | | chicken (Sample 7) |

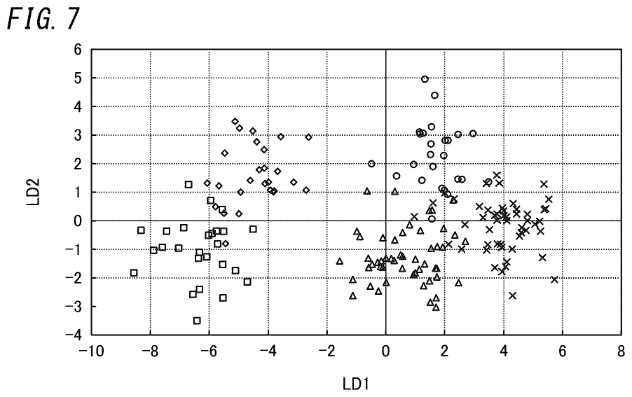

A discriminant and a decision boundary specified by the learned model were used to determine, based on the detection data obtained by using the sensor device for the volatile component released from each sample, to which groups respective samples belong. Specifically, the discriminant was used to specify a first discriminant coefficient and a second discriminant coefficient based on each detection data, and further, the decision boundary was used to determine to which group a combination of the first discriminant coefficient and the second discriminant coefficient belongs. Note that FIG. 7 is a scatter diagram of the detection data, where the abscissa (LD1) represents the first discriminant coefficient, and the ordinate (LD2) represents the second discriminant coefficient. The percentage of correct determinations was 92%.

As the embodiment and the examples clearly show, a food product spoilage determination method of a first aspect of the present disclosure includes trapping a volatile component released from a food product, acquiring, by using a detector (1), detection data on a plurality of components included in the volatile component, and when the food product is spoiled, determining, based on the detection data, at least one of a type of the food product or a degree of spoilage of the food product.

The first aspect enables the spoilage of the food product to be accurately determined.

In a second aspect of the present disclosure referring to the first aspect, the detection data includes information according to an amount of the plurality of components included in the volatile component.

The second aspect enables accuracy in determining the spoilage of the food product to be further enhanced.

In a third aspect of the present disclosure referring to the first or second aspect, the food product includes pork, and the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, 2-heptanone, and isobutyl isobutyrate.

The third aspect enables the accuracy of determination as to the spoilage of pork to be enhanced.

In a fourth aspect of the present disclosure referring to the third aspect, the plurality of components included in the volatile component include at least one of the 2-heptanone or the isobutyl isobutyrate.

The fourth aspect enables the accuracy of determination as to the spoilage of pork to be further enhanced.

In a fifth aspect of the present disclosure referring to the first or second aspect, the food product includes chicken, and the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, anisole, and styrene.

The fifth aspect enables the accuracy of determination as to the spoilage of chicken to be enhanced.

In a sixth aspect of the present disclosure referring to the fifth aspect, the plurality of components included in the volatile component include at least one of the anisole or the styrene.

The sixth aspect enables the accuracy of determination as to the spoilage of chicken to be further enhanced.

In a seventh aspect of the present disclosure referring to the first or second aspect, the food product includes pork and chicken, and the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, 2-heptanone, isobutyl isobutyrate, anisole, and styrene.

The seventh aspect enables the accuracy of determination as to the spoilage of the food product to be enhanced when the food product includes pork and chicken.

In an eighth aspect of the present disclosure referring to the seventh aspect, the plurality of components included in the volatile component include at least one of the 2-heptanone or the isobutyl isobutyrate and at least one of the anisole or the styrene.

The eighth aspect enables the spoilage of the pork and the spoilage of the chicken to be accurately determined also when the food product includes both the pork and the chicken.

In a ninth aspect of the present disclosure referring to any one of the first to eighth aspects, the detector (1) includes a gas sensor (2).

The ninth aspect enables accuracy in determining the spoilage of the food product to be further enhanced.

In a tenth aspect of the present disclosure referring to the ninth aspect, the gas sensor (2) is a sensor array including a plurality of sensor elements (Ax) having sensitivity characteristics different from each other.

The tenth aspect enables accuracy in determining the spoilage of the food product to be further enhanced.

In an eleventh aspect of the present disclosure referring to any one of the first to eighth aspects, the detector (1) is a gas chromatograph.

The eleventh aspect enables accuracy in determining the spoilage of the food product to be further enhanced.

In a twelfth aspect of the present disclosure referring to any one of the first to eleventh aspects, the determining is performed based on the detection data by using a learned model obtained by executing machine learning using training data.

The twelfth aspect enables accuracy in determining the spoilage of the food product to be further enhanced.

A food product spoilage determination system (5) according to a thirteenth aspect of the present disclosure includes: a detector (1) configured to detect a volatile component released from a food product to output an output signal; and a processor (50). The processor (50) includes: an acquirer (53) configured to acquire the output signal and generate, from the output signal, detection data on a plurality of components included in the volatile component; and a determining unit (55) configured to, when the food product is spoiled, make, based on the detection data, a determination as to at least one of a type of the food product or a degree of spoilage of the food product.

The thirteenth aspect enables the spoilage of the food product to be accurately determined.

In a fourteenth aspect of the present disclosure referring to the thirteenth aspect, the determining unit (55) is configured to make, based on the detection data, the determination by using a learned model obtained by executing machine learning using training data.

The fourteenth aspect enables accuracy in determining the spoilage of the food product to be further enhanced.

In a fifteenth aspect of the present disclosure referring to the fourteenth aspect, the determination includes a discriminant analysis based on the training data and the detection data.

The fifteenth aspect enables accuracy in determining the spoilage of the food product to be further enhanced.

In a sixteenth aspect of the present disclosure referring to any one of the thirteenth to fifteenth aspects, the detector (1) includes a gas sensor (2).

The sixteenth aspect enables accuracy in determining the spoilage of the food product to be further enhanced.

In a seventeenth aspect of the present disclosure referring to the sixteenth aspect, the gas sensor (2) is a sensor array including a plurality of sensor elements (Ax) having sensitivity characteristics different from each other.

The seventeenth aspect enables accuracy in determining the spoilage of the food product to be further enhanced.

In an eighteenth aspect of the present disclosure referring to any one of the thirteenth to seventeenth aspects, the food product spoilage determination system (5) further includes a display unit (57) configured to display a result of the determination.

The eighteenth aspect enables accuracy in determining the spoilage of the food product to be further enhanced.

In a nineteenth aspect of the present disclosure referring to any one of the thirteenth to eighteenth aspects, the food product spoilage determination system (5) configures a single device.

The nineteenth aspects enables the determination system (5) to be brought to a site where a food product is stored, thereby facilitating an on-site determination as to the spoilage of the food product.

In a twentieth aspect of the present disclosure referring to any one of the thirteenth to nineteenth aspects, the food product spoilage determination system (5) further includes a blower (15) configured to produce a current of air which transports the volatile component to the detector (1), and a rate of the current is greater than or equal to 10 mL/min and less than or equal to 3000 mL/min.

The twentieth aspect enables accuracy in determining the spoilage of the food product to be further enhanced.

### REFERENCE SIGNS LIST

1 Detector
15 Blower
2 Gas Sensor
5 Food Product Spoilage Determination System
53 Acquirer
55 Determining Unit
57 Display Unit
Ax Sensor Element

1. A food product spoilage determination method comprising:
trapping a volatile component released from a food product;
acquiring, by using a detector, detection data on a plurality of components included in the volatile component; and
when the food product is spoiled, determining, based on the detection data, at least one of a type of the food product or a degree of spoilage of the food product.

2. The food product spoilage determination method of claim 1, wherein the detection data includes information according to an amount of the plurality of components included in the volatile component.

3. The food product spoilage determination method of claim 1, wherein
the food product includes pork, and
the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, 2-heptanone, and isobutyl isobutyrate.

4. The food product spoilage determination method of claim 3, wherein
the plurality of components included in the volatile component include at least one of the 2-heptanone or the isobutyl isobutyrate.

5. The food product spoilage determination method of claim 1, wherein
the food product includes chicken, and
the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, anisole, and styrene.

6. The food product spoilage determination method of claim 5, wherein the plurality of components included in the volatile component include at least one of the anisole or the styrene.

7. The food product spoilage determination method of claim 1, wherein
the food product includes pork and chicken, and
the plurality of components included in the volatile component include at least one selected from the group consisting of 1-decanol, 2-butanone, ethyl 2-methylbutyrate, 2-pentanone, dimethyl disulfide, dimethyl trisulfide, ethyl tiglate, isobutyl alcohol, trimethylamine, 2-heptanone, isobutyl isobutyrate, anisole, and styrene.

8. The food product spoilage determination method of claim 7, wherein the plurality of components included in the volatile component include at least one of the 2-heptanone or the isobutyl isobutyrate and at least one of the anisole or the styrene.

9. The food product spoilage determination method of claim 1, wherein
the detector includes a gas sensor.

10. The food product spoilage determination method according to claim 9, wherein
the gas sensor is a sensor array including a plurality of sensor elements having sensitivity characteristics different from each other.

11. The food product spoilage determination method of claim 1, wherein
the detector is a gas chromatograph.

12. The food product spoilage determination method of claim 1, wherein
the determining is performed based on the detection data by using a learned model obtained by executing machine learning using training data.

13. A spoilage determination system comprising:
a detector configured to detect a volatile component released from a food product to output an output signal; and
a processor,
the processor including
an acquirer configured to acquire the output signal and generate, from the output signal, detection data on a plurality of components included in the volatile component and
a determining unit configured to, when the food product is spoiled, make, based on the detection data, a determination as to at least one of a type of the food product or a degree of spoilage of the food product.

14. The spoilage determination system of claim 13, wherein
the determining unit is configured to make, based on the detection data, the determination by using a learned model obtained by executing machine learning using training data.

15. The spoilage determination system of claim 14, wherein
the determination includes a discriminant analysis based on the training data and the detection data.

16. The spoilage determination system of claim 13, wherein the detector includes a gas sensor.

17. The spoilage determination system of claim 16, wherein
the gas sensor is a sensor array including a plurality of sensor elements having sensitivity characteristics different from each other.

18. The spoilage determination system of claim 13, further comprising
a display unit configured to display a result of the determination.

19. The spoilage determination system of claim 13, wherein
the spoilage determination system configures a single device.

20. The spoilage determination system of claim 13, further comprising
a blower configured to produce a current of air which transports the volatile component to the detector, wherein
a rate of the current is greater than or equal to 10 mL/min and less than or equal to 3000 mL/min.
